(19) United States
(12) Patent Application Publication
(10) Pub. No.: US 2019/0091496 A1
(43) Pub. Date: Mar. 28, 2019

(54) RESOURCE DEPLETION CALCULATION AND FEEDBACK FOR BREATHING EQUIPMENT

(71) Applicant: Blast Mask, LLC, Dallas, TX (US)

(72) Inventors: Justin C. Dickstein, Dallas, TX (US); Davis M. Denny, IV, Thorndale, TX (US); Khoa Nguyen Ahn Tran, Houston, TX (US); Patrick J. Griffin, McKinney, TX (US); Adam Mlynarczyk, Plano, TX (US)

(21) Appl. No.: 16/144,579

(22) Filed: Sep. 27, 2018

Related U.S. Application Data
(60) Provisional application No. 62/564,530, filed on Sep. 28, 2017.

(51) Int. Cl. A62B 9/00; A61M 16/00; A61M 16/10

(52) U.S. Cl. CPC A62B 9/006; A61M 2016/0039; A61M 16/107; A61M 16/024

(57) ABSTRACT

Methods, computer-readable media, and breathing equipment training devices for simulated resource depletion calculation. The breathing equipment training device includes a shell including an opening, a sensor connected to the shell, and a controller connected to the shell and operably connected to the sensor. The controller is configured to calculate, based on inputs from the sensor, a flowrate of air entering the breathing equipment training device through the opening of the shell and calculate an amount of depletion of a simulated resource for a duration of time based on the calculated flowrate of air entering the opening of the shell. The controller is configured to identify a current status of the simulated resource based on the calculated resource depletion amount and a prior status of the simulated resource identified prior to the duration of time and generate a feedback signal indicating the current status of the simulated resource.

### TECHNICAL FIELD

The present disclosure relates generally to breathing equipment. More particularly, the present disclosure relates to devices and methods for calculating and providing feedback on resource depletion for breathing equipment.

### BACKGROUND

When working in potentially hazardous environments, many types of people—firemen, for example—often use breathing protection devices, such as a self-contained breathing apparatus (SCBA), among other devices, to breathe. For example, oxygen supply may be depleted in a potentially hazardous environment and/or the air in a potentially hazardous environment may not be fit for breathing. Given the risk and potential hazards, individuals should be properly trained to operate their equipment, such as the SCBA, and understand its limitations before entering and/or working in such environments.

Breathing protection devices typically include depletable resources that provide protection in the form of modified breathing conditions for the person using the device. For example, SCBA devices include air tanks with clean air to breathe. Gas masks or respirators, such as air-purifying respirators (APRs) or chemical, biological, radiological, and nuclear (CBRN) masks, have filters that remove contaminates from the air. These resources are depletable. The amount of air in the tank is finite and the amount of contaminates that a filter can remove is limited.

Knowing when these depletable resources need to be replaced is important for safety. Running out of air in the tank or using up the filtration abilities of a mask while the user is still present in the potentially hazardous environment can be harmful. Traditionally, SCBA devices, for example, provide information about an amount of air remaining in the air tank to allow the air tank to be replaced or the user to exit the potentially hazardous environment to breath ambient air. This is usually accomplished using a pressure gauge that measures the amount of air pressure remaining in the tank.

### SUMMARY

Embodiments of the present disclosure provide a platform for calculating and providing feedback on resource depletion for breathing equipment

In an embodiment, a breathing equipment training device for simulated resource depletion calculation is provided. The breathing equipment training device includes a shell including an opening, a sensor connected to the shell, and a controller connected to the shell and operably connected to the sensor. The controller is configured to calculate, based on inputs from the sensor, a flowrate of air entering the breathing equipment training device through the opening of the shell and calculate an amount of depletion of a simulated resource for a duration of time based on the calculated flowrate of air entering the opening of the shell. The controller is configured to identify a current status of the simulated resource based on the calculated resource depletion amount and a prior status of the simulated resource identified prior to the duration of time and generate a feedback signal indicating the current status of the simulated resource.

In another embodiment, a method for simulated resource depletion calculation for a breathing equipment training device is provided. The method includes calculating, using a sensor of the breathing equipment training device, a flowrate of air entering the breathing equipment training device through an opening of the breathing equipment training device and calculating an amount of depletion of a simulated resource for a duration of time based on the calculated flowrate of air entering the opening of the breathing equipment training device. The method also includes identifying a current status of the simulated resource based on the calculated resource depletion amount and a prior status of the simulated resource identified prior to the duration of time and providing feedback indicating the current status of the simulated resource.

In yet another embodiment, a non-transitory, computer-readable medium comprising program code for simulated resource depletion calculation is provided. The program code, when executed by a controller, causes the controller to calculate, based on inputs from a sensor of a breathing equipment training device, a flowrate of air entering the breathing equipment training device through an opening of the breathing equipment training device and calculate an amount of depletion of a simulated resource for a duration of time based on the calculated flowrate of air entering the opening of the breathing equipment training device. The program code, when executed by the controller, further causes the controller to identify a current status of the simulated resource based on the calculated resource depletion amount and a prior status of the simulated resource identified prior to the duration of time and generate a feedback signal indicating the current status of the simulated resource.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates a perspective view of a breathing equipment training device in accordance with various embodiments of the present disclosure;

FIG. 2 illustrates a cross-sectional view of the breathing equipment training device illustrated in FIG. 1;

FIG. 3 illustrates a top view of the breathing equipment training device illustrated in FIG. 1;

FIG. 4 illustrates a block diagram of components for a resource depletion calculation and feedback system that can be included in a breathing equipment training device in accordance with various embodiments of the present disclosure;

FIG. 5 illustrates an example of electronic components included a resource depletion calculation and feedback system in accordance with various embodiments of the present disclosure;

FIG. 6 illustrates a mask for a SCBA which may be utilized in implementing various embodiments of the present disclosure;

FIG. 7 illustrates a pressure graph for calculating resource depletion in accordance with various embodiments of the present disclosure;

FIG. 8 illustrates a flowchart of a process for monitoring a resource status for a breathing equipment training device in accordance with various embodiments of the present disclosure; and

FIG. 9 illustrates a flowchart of a process for calculating and providing feedback on air tank depletion for a breathing equipment training device in accordance with various embodiments of the present disclosure.

### DETAILED DESCRIPTION

Embodiments of the present disclosure recognize and take into account that it would be advantageous to have systems and methods that take into account one or more of the issues discussed above, as well as possibly other issues, in order to more accurately simulate the conditions one would encounter in a potentially hazardous situation and to provide a trainee with feedback regarding the performance of his or her equipment. Various embodiments of the present disclosure recognize and take into account that, for safety reasons, people needing to use breathing equipment, such as, for example, firemen, construction workers, hazardous material response personnel, military personnel, underwater divers, etc., should first train with their equipment. For example, to preserve air supply, a SCBA utilizes on-demand breathing. This requires monitoring the remaining air supply in the SCBA tank so that an individual can avoid the risk of running out of fresh air in a potentially hazardous environment. In another example, filters for APRs or CBRN masks are rated to provide effective contaminate filtration percentages for a certain period of time based on a given flow rate of air through the filter (and other constants that are not controllable by the user, such as relative humidity).

Embodiments of the present disclosure recognize and take into account that proper feedback regarding remaining air or mask filtration ability would, in this example situation, assist in training users to control their breathing to more efficiently use their air supply or filtration ability and would help familiarize users with the lifespan of a tank of air or filter so that they can avoid getting trapped somewhere without breathable air. By mimicking the breathing resistance of an operational breathing device, monitoring air flow in the apparatus, and providing feedback to a user how much air would remain if an air tank was attached, breathing equipment training provided by embodiments of the present disclosure can provide many forms of emergency response training, among other activities, that is more cost-effective (refilling air tanks or replacing filter cartridges for training is expensive) and better simulates equipment performance.

Various embodiments of the present disclosure further recognize and take into account that the use of depletable resources, such as air tanks and filters, among other things, in the training of personnel to operate breathing equipment is costly. For example, training a person to properly control his or her breathing and to be aware of the remaining air in the breathing equipment can waste air in a tank when the ambient air is perfectly breathable. Each time a tank's air supply is depleted it must be refilled to repeat the activity and, over time, the refilling costs can become substantial. In another example, training with the masks for filtering out particles (e.g., APRs, gas masks, or CBRNE masks) may wear out the filtration mechanisms resulting in replacement costs for use in hazardous environments. Accordingly, various embodiments of the present disclosure provide breathing equipment training devices and methods that allow people to train to use breathing equipment without needing to have an air tank or wear out the actual protective gear. The use of various embodiments of the present disclosure to accurately estimate the rate of air consumption would allow people to train effectively with any breathing devices that are limited by, among other things, tank capacity or filter degradation rates such as SCBA tanks and filtration mechanisms used in APRs, gas masks, or CBRNE masks, respectively.

The different illustrative embodiments provide methods and devices for analyzing air flow through a training breathing apparatus and for providing feedback regarding various performance parameters of a breathing apparatus to simulate the use and limitations of a standard respiration-assistive system—which ordinarily may be costly to refill or replace—that would be used in a hazardous situation.

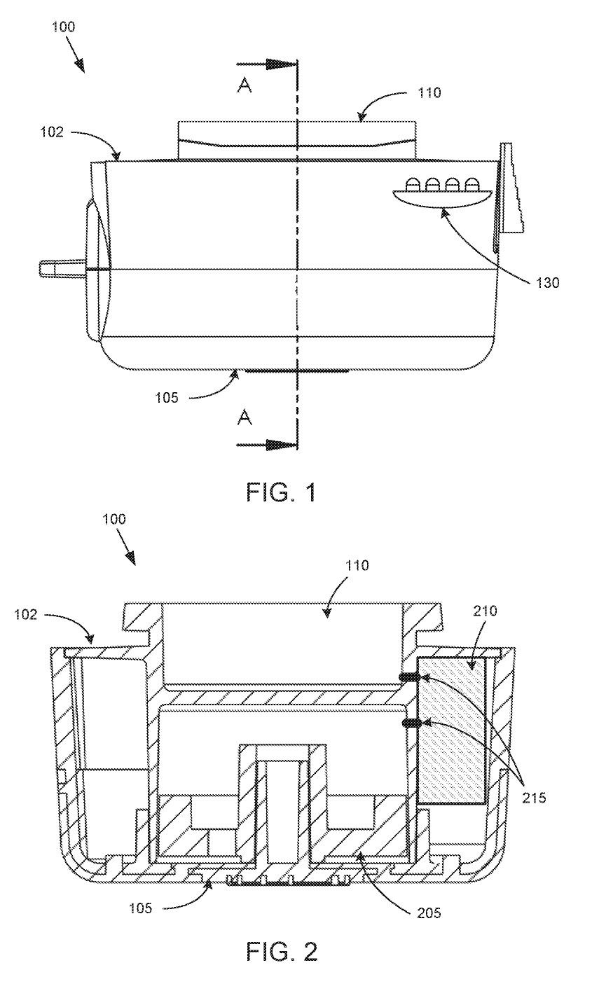

FIG. 1 illustrates a perspective view of a breathing equipment training device in accordance with various embodiments of the present disclosure. In this illustrative embodiment, breathing equipment training device 100 includes a cylindrically-shaped shell 102 with an opening 105 designed to allow air to flow into (for inhalation) and out (for exhalation) of a mask (e.g., mask 600 in FIG. 6) of an operator of breathing equipment, such as an SCBA or respirator. Breathing equipment training device 100 includes opening 110 designed to allow air flow out of (for inhalation) and into (for exhalation). For example, the training device 100 may take the place of a regulator (or filter) which is attached to the mask to regulate or otherwise control the flow of air into the mask. The shape and configuration of the breathing equipment training device 100 are for illustration only and the training device used in connection with the resource depletion calculation and feedback system 400 may take many forms. For example, each of the opening 105 and 110 may include any number of different openings of different shapes.

In this example, breathing equipment training device 100 also includes feedback lights 130 (e.g., LEDs) that provide feedback on the depletion of the air tank or filter. For example, the LEDs may be red, yellow, and green and may blink, flash, or steadily emit light to signal different amounts of resource depletion. As used herein, resource, when used in connection with a breathing equipment training device, means a depletable resource used with the actual device or system for which the wearer is being trained. For example, the resource may be air in a tank or a filter or filtration system for a respirator or gas mask.

FIG. 2 illustrates a cross-sectional view of the breathing equipment training device 100 illustrated in FIG. 1. In this illustrative embodiment, the breathing equipment training device 100 is seen opened along the cross-section denoted by line AA in FIG. 1. Inside of the shell 102, diaphragm 205 is located. The diaphragm 205 or valve covers the opening 105 and is made of a flexible material so as to impede or resist (but not completely block) the flow of air and other fluids through the opening 105. For example, the diaphragm 205 or valve may be made from rubber, plastic, polyurethane, a composite material, etc. In other embodiments, the diaphragm 205 could replicate the air resistance equivalent of a filter or filter cartridge (e.g., may be a fixed position filter) that approximates the effects of breathing through a gas mask or respirator.

Also located within the device 100 are electronic components 210 for the resource depletion calculation and feedback system 400 as discussed in greater detail below. One of the electronic components 210 is/are sensor(s) 215 placed at one or more points in the device to measure air flow. For example, breathing with the diaphragm 205 creates a significant change in air pressure between the different sections of the device 100 while someone draws air through the device 100. As discussed in greater detail, the sensor(s) 215, such as pressure sensors placed, within the device 100 at various points record these differences in order to calculate breath time and volume, among possibly other data.

In this manner, when attached to a mask, the breathing equipment training device 100 impedes or resists the flow of air into the mask, simulating usage of breathing equipment using on-demand breathing. Different types of diaphragms or valves having different levels of flexibility or resistance to air may be used to simulate different levels of inhalation force that may be required to operate the on-demand breathing equipment.

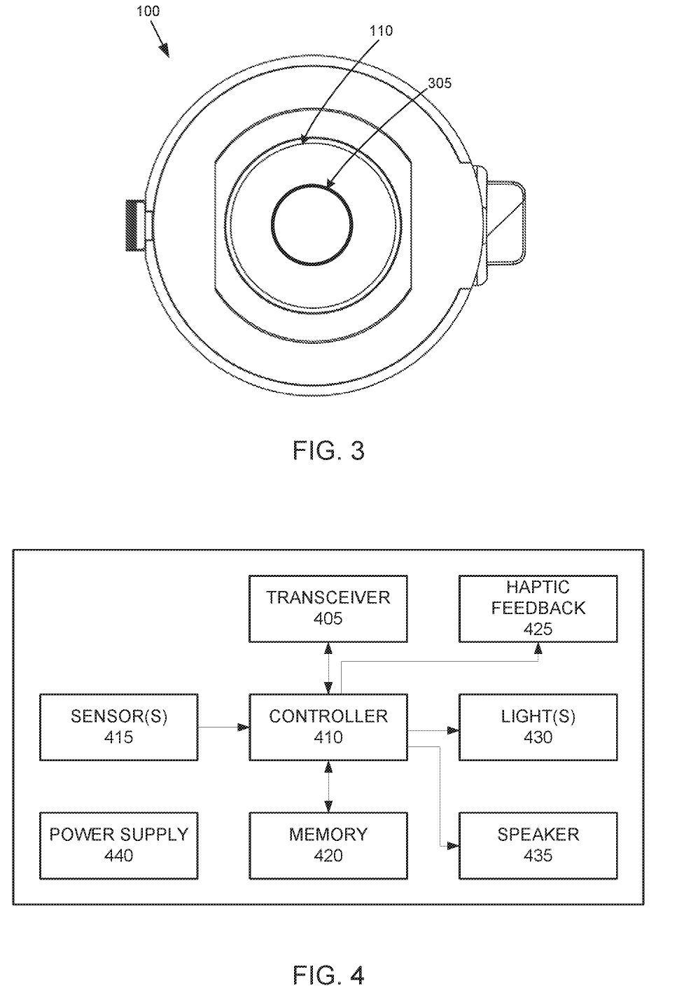

FIG. 3 illustrates a top view of the breathing equipment training device 100 illustrated in FIG. 1. As illustrated, opening 110 allows for air flow into and out of the device 100. Within the opening 110 is an orifice 305 that has a smaller diameter compared to the larger airway defined by openings 105 and 110. This smaller area leads to greater pressure changes when air is inhaled in due to the increased velocity of the air through the opening. Sensor(s) 215 in and/or around the orifice 305 will measure the air pressure during inhalation so that velocity through a known area can be computed to give a volumetric flow rate for each breath taken. In various embodiments, the sensor(s) 215 are positioned proximate to the opening 110 and output voltages that vary with pressure so that a microcontroller can collect the data, correct for “noise,” calculate volumetric flow rate in the device, and provide informative LED and/or haptic feedback regarding various flow-dependent parameters.

FIG. 4 illustrates a block diagram of components for a resource depletion calculation and feedback system 400 that can be included in a breathing equipment device in accordance with various embodiments of the present disclosure. The embodiment of the system 400 illustrated in FIG. 4 is for illustration only. System 400 can come in a wide variety of configurations, and FIG. 4 does not limit the scope of this disclosure to any particular system implementation. As shown in FIG. 4, the system 400 includes a transceiver 405; a controller 410; a sensor(s) 415; memory 420; feedback devices, which can include, in this embodiment, one or more of haptic feedback device 425, light(s) 430, and speaker 435; and a power supply 440.

The transceiver 405 supports communications with other systems or devices. The transceiver 405 may support communications through any suitable physical or wireless communication link(s). For embodiments utilizing wired communication, the transceiver 405 may be a Universal Serial Bus (USB) port or network interface card used, for example, to program the controller 410 or communicate resource status information to an external feedback device. For embodiments utilizing wireless communication, the transceiver 405 may receive and/or transmit an RF signal via one or more antennas using a variety of wireless communication protocols, (e.g., Bluetooth, Wi-Fi, cellular, LTE communication protocols etc.), for example, to communicate resource status information to another device, such as, for example a computer in a command center, a portable/handheld feedback device, mobile phone, etc.

The controller 410 can include one or more controllers or other processing devices and execute instructions stored in the memory 420 in order to control the overall operation of the system 400. In various embodiments, the controller 410 instructions resident in the memory 420 to calculate resource depletion based on inputs received from sensor(s) 415 and to provide feedback to an operator using one or more of the feedback devices 425-435 as discussed in greater detail below. The controller 410 may include any suitable number(s) and type(s) of controllers or other devices in any suitable arrangement. Example types of controller 410 include microcontrollers, microcontrollers, digital signal controllers, field programmable gate arrays, application specific integrated circuits, and discreet circuitry.

The sensor(s) 415 in the system 400 can be any type of sensor for monitoring a status of a depletable resource used in connection with breathing equipment. In various embodiments, the sensor(s) 415 may be any type of sensor that can be used to calculate an air flow rate, for example, when used in connection with an opening of known size. For example, without limitation, the sensor(s) 415 can be one or more pressure sensors, turbine sensors, mass flow sensors, spirometers, etc.

The feedback devices 425-435 provide feedback to the operator of the device 100 about the status (e.g., remaining quantity or quality) of the resource. The haptic feedback device 425 provides tactile feedback, the light(s) 430 provide visual feedback, and the speaker 435 provides audible feedback. For example, in one embodiment, the haptic feedback device 425 may be a rumble or vibrating motor that activates when the resource is calculated to be below a certain level (e.g., ⅓ or less of the resource remaining). In some embodiments, the vibration frequency of the vibrations produced by the haptic feedback device 425 may increase or decrease based on the amount of resource remaining. For example, in one embodiment, the light(s) 430 may include multiple LEDs with a green light shown from full tank to half tank, a yellow light shown from a half tank to a third tank, and a red light shown from a third tank to empty. In some embodiments, the light(s) (e.g., the last red light) may flash when resource quantity or quality is below some threshold (e.g., signaling a critically low level of resource remaining). For example, in some embodiments, the speakers 435 may output a tone or verbal indication of resource level or may signal an alarm based on the amount of resource depletion calculated by the system 400.

System 400 further includes power supply 440 to provide power to the various components in the system through electrical connections, which are not shown in FIG. 4 for simplicity but are inherently present. The power supply 440 can be a battery, such as a replaceable or rechargeable (e.g., via a port such as USB port or other types of charging ports) battery to provide power for the system.

Although FIG. 4 illustrates one example of system 400, various changes may be made to FIG. 4. For example, various components in FIG. 4 could be combined, further subdivided, or omitted and additional components could be added according to particular needs. As a particular example, the controller 410 could be divided into multiple controllers, such as one or more central processing units (CPUs), and/or may have the memory 420 integrated into the controller 410. In another example, only one of or any combination the feedback devices 425-435 may be included in the system 400.

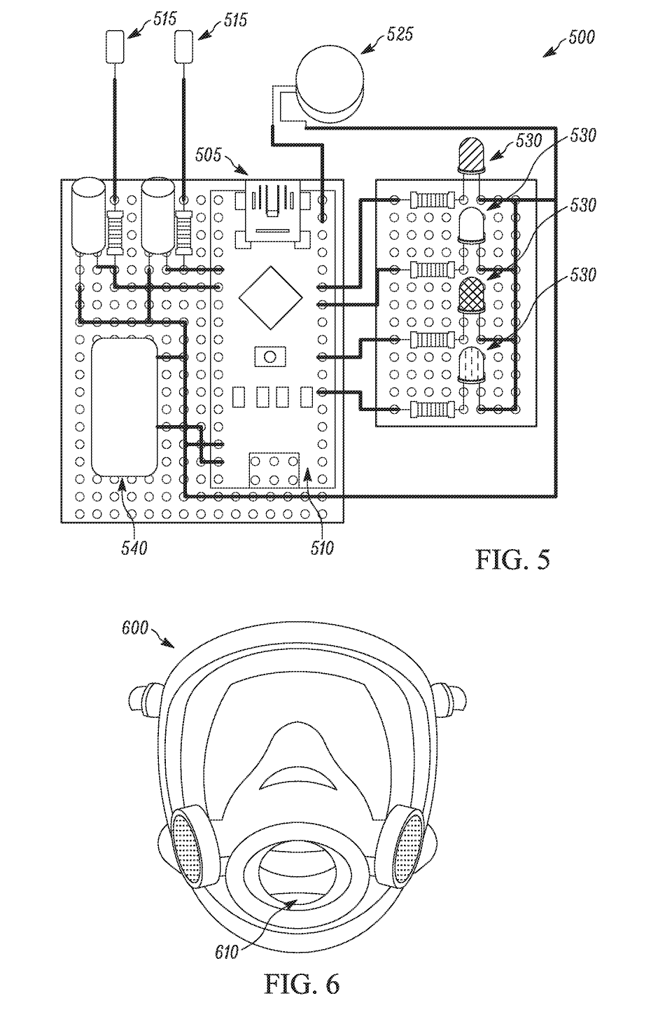

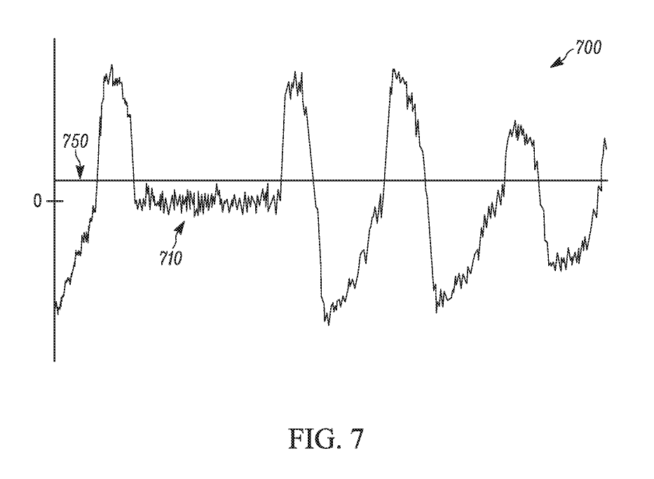

FIG. 5 illustrates an example of electronic components included an example resource depletion calculation and feedback system 500 in accordance with various embodiments of the present disclosure. In this embodiment, pressure sensors 515 send a voltage output to a controller 510 to perform calculations about how much air is being inhaled in an iterative manner. In this example, the pressure measurements are used to calculate air velocity through equations describing Bernoulli's relation between velocity and pressure. With the air velocity known, the flow rate is then be calculated by using the known dimensions of the apparatus through which the air is flowing. Breath time is measured as shown in FIG. 7 in order to calculate the volume of air used in that particular breath. At the end of each program loop, various output devices, such as the lights 530 or the haptic device 525, are activated depending on the new state of certain dependent variables.

FIG. 6 illustrates a mask 600 for a SCBA that may be utilized in implementing various embodiments of the present disclosure. The mask 600 is designed to be worn over the head and face of the operator to protect the eyes, nose, and mouth of the operator in hazardous environments and/or in environments where breathable ambient air is not present. In this illustrative example, mask 600 includes a breathing opening 605 matched that would usually be connected to a regulator and, by extension, an air tank. However, the breathing equipment training device 100 of the present disclosure can be substituted for a regulator and the resource depletion calculation and feedback system of the present disclosure can provide the user with the same or similar experience on feedback for the status of the air tank.

FIG. 7 illustrates a pressure graph for calculating resource depletion in accordance with various embodiments of the present disclosure. The pressure difference between sensors in this example spikes during inhalation as the orifice 305 causes a change in pressure. During exhalation, the pressure change reading falls below zero as air is sent the opposite direction. In various embodiments, the air flow associated with exhalation is disregarded as not relevant towards the calculation of resource depletion. However, in some embodiments where the amount of exhalation is a factor, for example, such as for measuring filter wear, the amount or volume of exhalation may also be calculated similarly to the calculation of inhalation volume. Line 705 is an example threshold level set in response to the noise level in the data. In this example, only the pressure readings that reach above the line are used to calculate the volumetric flow rate of the breath. This threshold could be set to account for data noise 710 or it can be adjusted to account for the resistance of a regulator or other breathing device if the physical elements of the training apparatus are not accounted for or accurately simulated elsewhere within the device. Additionally, the measured pressure values may be averaged over time (e.g., using a digital two-step averaging filter such that every 50 data points correspond to one actual data point in volume calculation) to further normalize the received inputs.

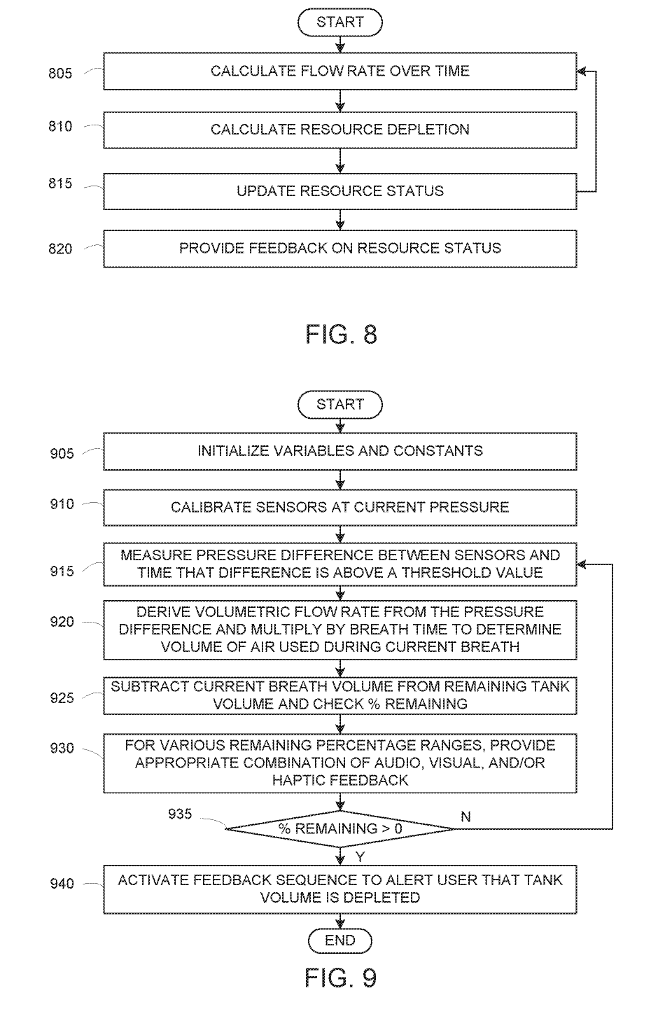

FIG. 8 illustrates a flowchart of a process for monitoring a resource status for a breathing equipment training device in accordance with various embodiments of the present disclosure. By way of example, the process depicted in FIG. 8 can be implemented by the system 400 or controller 410 in FIG. 4 or the system 500 in FIG. 5 (collectively or individually referred to as “the system”) to provide simulated resource depletion calculation for a breathing equipment training device.

In these embodiments, the process begins with the system calculating flow rate over time (step 805). For example, in step 805, the system calculates a flowrate of air entering the breathing equipment training device through an opening of the breathing equipment training device for some particular duration of time. These calculations may be performed using sensor inputs for calculating flow rates, such as pressure values or turbine speed. In various embodiments, the sensor is a single pressure sensor positioned proximate to the opening. In some embodiments, the sensor is two pressure sensors (e.g., sensors 215) that are positioned on opposite sides of an orifice (e.g., orifice 305) in the opening (e.g., opening 110). In various embodiments, the resource status being monitored is a simulated resource, in other words, not an actual resource that is part of the breathing equipment, but a depletable resource intended to be used with or for the breathing equipment in live or non-training situations. In some embodiments, the simulated resource is a volume of air in an air tank and the amount of depletion that is calculated using the flow rate is an estimate of a simulated reduction in the quantity of air in the simulated air tank as a result of use for the duration of time. In other embodiments, the simulated resource is an ability of an air filter to filter ambient air and the amount of depletion that is calculated using the flow rate is an estimate of a simulated reduction in ability of the simulated air filter to filter the ambient air as a result of use for the duration of time.

Thereafter, the system calculates resource depletion (step 810). For example, in step 810, the system determines an amount of the simulated resource depleted for the duration of time based on known values associated with the resource and the calculated flow rate. In some embodiments, the known values may be the known dimensions of the openings 105 and 110 and/or orifice 305 of the breathing device 100 that when combined (e.g., multiplied) by the current flow rate yields a current inhalation volume which is summed overtime to calculate the current resource depletion level for the monitored duration of time.

In other embodiments, the volume of air inhaled may be calculated similarly as above but the known values may be a volume of air that a filter is rated for, a percentage of contaminates per volume of air in some actual or potentially hazardous environment, and/or an amount of contaminates a filter can filter before needing replacement. In these embodiments, the system calculates the amount of air and/or contaminates that are received by the filter to determine the amount of depletion of the filter resources.

In some examples of these embodiments, filters used in certain APRs or CNRN masks are rated to have a minimum effectiveness time length against contaminants and concentrations based on the flow rate of air entering the filter (and other constants not controllable by the user such as relative humidity and temperature). For example, a “CAP 1” filter may be 99% effective for 15 minutes at a flow rate of 65 liters per minute for a given contaminant concentration, temperature, and relative humidity. However, increasing the flow rate to 100 liters per minute may decrease the filtration ability to 5 minutes for the same contaminant concentration, temperature, and relative humidity. Using (i) preset standard, configurable, or dynamically measured values, such as, for example, using additional temperature, humidity, and/or contaminate concentration sensors beyond the pressure sensor, for the constants (e.g., contaminant concentration, temperature, and relative humidity), (ii) the defined relationship between effective filter time and flow rate, and (iii) the above calculated flow rate for the duration of time, the system calculates the amount of the resource depleted during the monitored duration of time (e.g., the reduction in the effective filtration time remaining for the filter or percent reduction in time based on a predefined standard amount of effective filtration time).

The system then updates resource status (step 815). For example, in step 815, the system identify a current status of the simulated resource based on the calculated resource depletion amount and a prior status of the simulated resource identified prior to the duration of time. For example, the system subtracts the amount of depletion from the initial or preceding simulated resource status to determine the current status of the resource. The system repeats these steps 805-815 iteratively to continue to monitor and update the status of the resource. For example, the depletion may be calculated and/or the resource status updated based on a fixed frequency, based on measured breathing cycles, or any other suitable timing.

Thereafter, the system provides feedback on resource status (step 820). For example, in step 820, the system may provide feedback in the form of lights, sound, and/or haptics as discussed above. In one example, the system may determine to, in response to determining that the current status of the simulated resource drops below a threshold status (e.g., providing visual feedback using feedback lights of the breathing equipment training device (e.g., transition between a green, yellow, or red light to indicate the amount of the resource remaining). In another example, the system may provide haptic feedback, such as by providing a vibration once the current status of the simulated resource drops below a threshold status and possibly increasing the frequency and/or intensity of the vibration as the current status of the simulated resource decreases. In another example, the system may provide audio feedback, such as by providing a chirping or bell sound once the current status of the simulated resource drops below a threshold status and possibly increasing the frequency and/or volume of the sound as the current status of the simulated resource decreases. In another example, the system may, in response to determining that the current status of the simulated resource drops below a second threshold status, provide some combination of two or more of visual, audio, and haptic feedback using the feedback lights, speaker, and/or haptic feedback device of breathing equipment training device, respectively (e.g., once the resource is nearly out, the system may both flash red lights and provide vibration or sound to simulate the near expiration of the resource). The process ends when the system is powered off or once the resource has been calculated to be fully depleted.

FIG. 9 illustrates a flowchart of a process for calculating and providing feedback on air tank depletion for a breathing equipment training device in accordance with various embodiments of the present disclosure. By way of example, the process depicted in FIG. 9 can be implemented by the system 400 or controller 410 in FIG. 4 or the system 500 in FIG. 5 (collectively or individually referred to as “the system”). The process depicted in FIG. 9 is one embodiment of the process illustrated in FIG. 8. In one embodiment, FIG. 9 illustrates a high-level depiction of various components of a logical code progression for an example implementation for calculating and providing feedback on air tank depletion utilizing the components illustrated in FIG. 5.

In these embodiments, the process begins with the system initialing variables and constants (step 905). For example, in step 905, the system may run a setup process to set up all sensors and variables, which may include identifying the initial starting value for the amount of air in the virtual or actual tank, run the LEDs to illustrate the system turning on and calibrating. Thereafter, the system calibrates sensors at current pressure (step 910). For example, in step 910, if using two pressure sensors, the system calibrates the sensors to each other so that each sensor is reading a same value at the beginning.

Next, the system moves to a measurement and feedback loop for steps 915-935. The system then measures pressures difference between sensors and time that difference is above a threshold value (step 915). For example, in one embodiment, in step 915, the system acquires voltage values from the pressure sensors (e.g., differential voltage values) and converts the voltage values into pressure values (e.g., as illustrated in FIG. 7) and uses a digital two-step averaging filter such that every 50 data points correspond to one actual data point in volume calculation. This stem may be implemented within code as a combination of two for loops that take values from the sensors then take the averages to get the desired data point.

Thereafter, the system derives volumetric flow rate from the pressure difference and multiply by breath time to determine the volume of air used during current breath (step 920). For example, in step 920, the system calculates the flow rate based on the pressure difference between the two pressure sensors then, using timers within this loop, the actual volume inhaled at each code iteration is calculated by multiplying flow rate by time. The system then subtracts current breath volume from remaining tank volume and checks percent remaining (step 925). For example, in step 925, the system subtracts the calculated volume inhaled from the initial tank volume or prior tank volume from a previous iteration of the loop. Thereafter, for various remaining percentage ranges, the system provides the appropriate combination of audio, visual, and/or haptic feedback (step 930). For example, in step 930, the system may provide feedback using one or more of the feedback devices 425-435 in any of the manners discussed above.

The system then determines whether the percent remaining is greater than zero (step 935). If so, the system returns to step 915 and continues to calculate and update depletion and provide appropriate feedback in an iterative manner in the measurement and feedback loop. When the percent remaining is zero, the system then activates a feedback sequence to alert a user that tank volume is depleted (step 940) with the process ending thereafter. For example, in step 940, the system may flash the LED lights and stop previous haptic feedback.

Although FIGS. 8 and 9 illustrate examples of processes for monitoring a resource status for a breathing equipment training device in accordance with various embodiments of the present disclosure and calculating and providing feedback on resource depletion for a breathing equipment training device in accordance with various embodiments of the present disclosure, respectively, various changes could be made to FIGS. 8 and 9. For example, while shown as a series of steps, various steps in each figure could overlap, occur in parallel, occur in a different order, or occur multiple times. In another example, steps may be omitted or replaced by other steps.

Embodiments of the present disclosure also include a method of training to use breathing equipment. In addition to the description above, the method includes attaching the breathing equipment training device 100 to a mask, e.g., mask 600 of breathing equipment such as a SCBA or respirator. The method further includes breathing through the mask 60 and the breathing equipment training device 100 to train for the on-demand breathing experienced using certain types of breathing equipment. Using this method, a system such as that described in FIG. 4 may be used to determine the air flow through the mask and how much each breath would drain from a hypothetical air tank. The system would provide feedback such that users would, in addition to the physical simulation of breathing through a functional mask, also be shown the oxygen levels that would be available to them if a real air tank were attached.

Moreover, the various figures and embodiments used to describe the principles of the present disclosure in this patent document are by way of illustration only and should not be construed in any way to limit the scope of the present disclosure. Those skilled in the art will understand that the principles of the present disclosure may be implemented in any type of suitably arranged device or system. Other such embodiments may resemble, relate to, or be used with, but are in no way limited to, CBRN gas masks, SCUBA gear, or CPR training equipment. In addition, the present disclosure should not be limited to analysis of bulk air flow, but may also be used with sensors that monitor, among other things, the flow of specific particles or chemicals such as, for example, carbon dioxide or carbon monoxide to provide feedback on accumulated intake or output of specific compounds. Moreover, while various embodiments are discussed in connection with training, any of the resource depletion calculation and/or feedback embodiments disclosed herein can be utilized in connection with actual usage of the equipment in addition to or instead of the traditional resource monitoring and/or feedback components for the actual equipment. For example, the resource depletion calculation and feedback system 400 can be used to augment or replace the air tank feedback system of a SCBA or SCUBA system and/or provide status information about a remaining quality of a filter or filter system included in a respirator or gas mask.

One embodiment provides a breathing equipment training device for simulated resource depletion calculation is provided. The breathing equipment training device includes a shell including an opening, a sensor connected to the shell, and a controller connected to the shell and operably connected to the sensor. The controller is configured to calculate, based on inputs from the sensor, a flowrate of air entering the breathing equipment training device through the opening of the shell and calculate an amount of depletion of a simulated resource for a duration of time based on the calculated flowrate of air entering the opening of the shell. The controller is configured to identify a current status of the simulated resource based on the calculated resource depletion amount and a prior status of the simulated resource identified prior to the duration of time and generate a feedback signal indicating the current status of the simulated resource.

Another embodiment provides a method for simulated resource depletion calculation for a breathing equipment training device is provided. The method includes calculating, using a sensor of the breathing equipment training device, a flowrate of air entering the breathing equipment training device through an opening of the breathing equipment training device and calculating an amount of depletion of a simulated resource for a duration of time based on the calculated flowrate of air entering the opening of the breathing equipment training device. The method also includes identifying a current status of the simulated resource based on the calculated resource depletion amount and a prior status of the simulated resource identified prior to the duration of time and providing feedback indicating the current status of the simulated resource.

Another embodiment provides a non-transitory, computer-readable medium comprising program code for simulated resource depletion calculation is provided. The program code, when executed by a controller, causes the controller to calculate, based on inputs from a sensor of a breathing equipment training device, a flowrate of air entering the breathing equipment training device through an opening of the breathing equipment training device and calculate an amount of depletion of a simulated resource for a duration of time based on the calculated flowrate of air entering the opening of the breathing equipment training device. The program code, when executed by the controller, further causes the controller to identify a current status of the simulated resource based on the calculated resource depletion amount and a prior status of the simulated resource identified prior to the duration of time and generate a feedback signal indicating the current status of the simulated resource.

In any of the above examples and embodiments, the sensor is a pressure sensor positioned proximate to the opening and the controller or method is configured to calculate the flowrate of the air entering the opening of the shell using inputs from the pressure sensor positioned proximate to the opening.

In any of the above examples and embodiments, the breathing equipment training device includes a second pressure sensor and the pressure sensors are positioned on opposite sides of an orifice in the opening.

In any of the above examples and embodiments, the breathing equipment training device includes feedback lights and the controller or method is configured to, in response to a determination that the current status of the simulated resource drops below a first threshold status, generate the feedback signal to provide visual feedback using the feedback lights.

In any of the above examples and embodiments, the breathing equipment training device includes at least one of a haptic feedback device and a speaker and the controller or method is configured to, in response to a determination that the current status of the simulated resource drops below a second threshold status, generate the feedback signal to provide (i) visual feedback using the feedback lights and (ii) haptic feedback using the haptic feedback device or audio feedback using the speaker.

In any of the above examples and embodiments, the simulated resource is a quantity of air in an air tank and the calculated amount of depletion is an estimate of a simulated reduction in the quantity of air in the simulated air tank as a result of use for the duration of time.

In any of the above examples and embodiments, the simulated resource is an ability of an air filter to filter ambient air and the calculated amount of depletion is an estimate of a simulated reduction in ability of the simulated air filter to filter the ambient air as a result of use for the duration of time.

It may be advantageous to set forth definitions of certain words and phrases used throughout this patent document. The terms “couple” and “connect” and their derivatives refer to any direct or indirect connection between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer-readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer-readable medium” includes any type of medium capable of being accessed by a computer, such as read-only memory (ROM), random access memory (RAM), a hard disk drive, a compact disc (CD), a digital video disc (DVD), or any other type of memory. A “non-transitory” computer-readable medium excludes wired, wireless, optical, or other communication links that transport transitory electrical or other signals. A non-transitory computer-readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases. Although the present disclosure has been described with an exemplary embodiment, various changes and modifications may be suggested to one skilled in the art. It is intended that the present disclosure encompasses such changes and modifications as fall within the scope of the appended claims.

None of the description in this application should be read as implying that any particular element, step, or function is an essential element that must be included in the claim scope. The scope of the patented subject matter is defined only by the claims. Moreover, none of the claims is intended to invoke 35 U.S.C. § 112(f) unless the exact words “means for” are followed by a participle.

What is claimed is:

1. A breathing equipment training device for simulated resource depletion calculation, the breathing equipment training device comprising:
a shell including an opening;
a sensor connected to the shell; and
a controller connected to the shell and operably connected to the sensor, the controller configured to:
calculate, based on inputs from the sensor, a flowrate of air entering the breathing equipment training device through the opening of the shell;
calculate an amount of depletion of a simulated resource for a duration of time based on the calculated flowrate of air entering the opening of the shell;
identify a current status of the simulated resource based on the calculated resource depletion amount and a prior status of the simulated resource identified prior to the duration of time; and
generate a feedback signal indicating the current status of the simulated resource.

2. The breathing equipment training device of claim 1, wherein:
the sensor is a pressure sensor positioned proximate to the opening, and
the controller is configured to calculate the flowrate of the air entering the opening of the shell using inputs from the pressure sensor positioned proximate to the opening.

3. The breathing equipment training device of claim 2, further comprising:
a second pressure sensor,
wherein the pressure sensors are positioned on opposite sides of an orifice in the opening.

4. The breathing equipment training device of claim 1, wherein:
the simulated resource is a quantity of air in an air tank,
the calculated amount of depletion is an estimate of a simulated reduction in the quantity of air in the simulated air tank as a result of use for the duration of time.

5. The breathing equipment training device of claim 1, wherein:
the simulated resource is an ability of an air filter to filter ambient air, and
the calculated amount of depletion is an estimate of a simulated reduction in ability of the simulated air filter to filter the ambient air as a result of use for the duration of time.

6. The breathing equipment training device of claim 1, further comprising:
feedback lights,
wherein the controller is configured to, in response to a determination that the current status of the simulated resource drops below a first threshold status, generate the feedback signal to provide visual feedback using the feedback lights.

7. The breathing equipment training device of claim 6, further comprising:
at least one of a haptic feedback device and a speaker,
wherein the controller is configured to, in response to a determination that the current status of the simulated resource drops below a second threshold status, generate the feedback signal to provide (i) visual feedback using the feedback lights and (ii) haptic feedback using the haptic feedback device or audio feedback using the speaker.

8. A method for simulated resource depletion calculation for a breathing equipment training device, the method comprising:
calculating, using a sensor of the breathing equipment training device, a flowrate of air entering the breathing equipment training device through an opening of the breathing equipment training device;
calculating an amount of depletion of a simulated resource for a duration of time based on the calculated flowrate of air entering the opening of the breathing equipment training device;
identifying a current status of the simulated resource based on the calculated resource depletion amount and a prior status of the simulated resource identified prior to the duration of time; and
providing feedback indicating the current status of the simulated resource.

9. The method of claim 8, wherein:
the sensor is a pressure sensor positioned proximate to the opening, and
calculating the flowrate of the air entering the opening of the breathing equipment training device comprises calculating the flowrate using inputs from the pressure sensor positioned proximate to the opening.

10. The method of claim 9, wherein:
the breathing equipment training device includes a second pressure sensor, and
the pressure sensors are positioned on opposite sides of an orifice in the opening.

11. The method of claim 8, wherein:
the simulated resource is a quantity of air in an air tank,
the calculated amount of depletion is an estimate of a simulated reduction in the quantity of air in the simulated air tank as a result of use for the duration of time.

12. The method of claim 8, wherein:
the simulated resource is an ability of an air filter to filter ambient air, and
the calculated amount of depletion is an estimate of a simulated reduction in ability of the simulated air filter to filter the ambient air as a result of use for the duration of time.

13. The method of claim 8, wherein providing feedback indicating the current status of the simulated resource further comprises:
in response to determining that the current status of the simulated resource drops below a first threshold status, providing visual feedback using feedback lights of the breathing equipment training device.

14. The method of claim 13, wherein providing feedback indicating the current status of the simulated resource further comprises:
in response to determining that the current status of the simulated resource drops below a second threshold status, providing (i) visual feedback using the feedback lights and (ii) haptic feedback using a haptic feedback device of the breathing equipment training device or audio feedback using a speaker of the breathing equipment training device.

15. A non-transitory, computer-readable medium comprising program code for simulated resource depletion calculation, the program code, when executed by a controller, causes the controller to:
calculate, based on inputs from a sensor of a breathing equipment training device, a flowrate of air entering the breathing equipment training device through an opening of the breathing equipment training device;
calculate an amount of depletion of a simulated resource for a duration of time based on the calculated flowrate of air entering the opening of the breathing equipment training device;
identify a current status of the simulated resource based on the calculated resource depletion amount and a prior status of the simulated resource identified prior to the duration of time; and
generate a feedback signal indicating the current status of the simulated resource.

16. The computer-readable medium of claim 15, wherein:
the sensor is a pressure sensor positioned proximate to the opening, and
the program code to calculate the flowrate of the air entering the opening of the breathing equipment training device comprises program code, that when executed by the controller, causes the controller to calculate the flowrate using inputs from the pressure sensor positioned proximate to the opening.

17. The computer-readable medium of claim 16, wherein:
the breathing equipment training device includes a second pressure sensor, and
the pressure sensors are positioned on opposite sides of an orifice in the opening.

18. The computer-readable medium of claim 15, wherein:
the simulated resource is a quantity of air in an air tank,
the calculated amount of depletion is an estimate of a simulated reduction in the quantity of air in the simulated air tank as a result of use for the duration of time.

19. The computer-readable medium of claim 15, wherein:
the simulated resource is an ability of an air filter to filter ambient air, and
the calculated amount of depletion is an estimate of a simulated reduction in ability of the simulated air filter to filter the ambient air as a result of use for the duration of time.

20. The computer-readable medium of claim 15, wherein the program code to generate the feedback signal comprises program code, that when executed by the controller, causes the controller to:
in response to a determination that the current status of the simulated resource drops below a first threshold status, generate the feedback signal to provide visual feedback using feedback lights of the breathing equipment training device; and
in response to a determination that the current status of the simulated resource drops below a second threshold status, generate the feedback signal to provide (i) visual feedback using the feedback lights and (ii) haptic feedback using a haptic feedback device of the breathing equipment training device or audio feedback using a speaker of the breathing equipment training device.
